(19) United States
(12) Patent Application Publication
(10) Pub. No.: US 2014/0046188 A1
(43) Pub. Date: Feb. 13, 2014

(54) System and Method for Ultrasonic Diagnostics

(71) Applicants: Jesse T. Yen, La Crescenta, CA (US); Harry L. Tarnoff, Sherman Oaks, CA (US); Mark H. Wittman, Los Angeles, CA (US); Richard P. Koffler, Pacific Palisades, CA (US)

(72) Inventors: Jesse T. Yen, La Crescenta, CA (US); Harry L. Tarnoff, Sherman Oaks, CA (US); Mark H. Wittman, Los Angeles, CA (US); Richard P. Koffler, Pacific Palisades, CA (US)

(21) Appl. No.: 13/961,676

(22) Filed: Aug. 7, 2013

Related U.S. Application Data
(60) Provisional application No. 61/680,469, filed on Aug. 7, 2012.

(51) Int. Cl. A61B 8/00

(52) U.S. Cl. CPC A61B 8/4427
USPC 600/444; 600/447

(57) ABSTRACT

A system for ultrasound imaging including an ultrasound transducer array housed in a single patch and comprising a plurality of ultrasound sensor elements and associated electronic; a beamformer electrically coupled to the ultrasound transducer array patch for combining outputs of said ultrasound sensor elements; and a CPU electrically coupled to the beamformer and configured to receive the combined outputs of said ultrasound sensor elements, perform image processing and analytics and generate an image or metrics to be displayed on a remote display screen.

### CROSS-REFERENCE TO RELATED APPLICATIONS

This Patent Application claims the benefits of U.S. Provisional Patent Application Ser. No. 61/680,469, filed on Aug. 7, 2012 and entitled “System and Method for Ultrasonic Diagnostics,” the entire content of which is hereby expressly incorporated by reference.

### FIELD OF THE INVENTION

The present invention relates generally to electronic imaging; and more particularly to a system and method for ultrasonic diagnostics of subjects.

### BACKGROUND

Ultrasound, which is an oscillating sound pressure wave, has applications in many different fields. For example, ultrasonic systems are used to detect objects and measure distances and ultrasonic imaging is used in medicine to produce images of, for example subject's internal organs, such as a heart. Ultrasound is also utilized in testing of products and structures, to detect invisible flaws. Other industrial applications include cleaning, mixing of some substances, and accelerating chemical processes, or changing the chemical properties of substances.

Medical ultrasound is popular because of its relative low cost and radiation-free operation. Portable handheld ultrasound systems that provide greater access to ultrasound diagnostic techniques have recently become available. While smaller and less expensive than their cart-bound ancestors, these portable handheld systems still follow the commonly used approach to diagnostic ultrasound devices consisting of a singular handheld probe connected with a cable to the control unit containing the system's front-end electronics, and a back-end computer with its user interface and display. Some of these portable handheld systems connect to networks and provide remote access to the ultrasound images.

### SUMMARY

In some embodiments, the present invention is a system for ultrasound imaging. The system includes an ultrasound transducer array housed in a single patch and comprising a plurality of ultrasound sensor elements and associated electronic; a beamformer electrically coupled to the ultrasound transducer array patch for combining outputs of said ultrasound sensor elements; and a central processing unit (CPU) electrically coupled to the beamformer and configured to receive the combined outputs of said ultrasound sensor elements, perform image processing and analytics and generate images and metrics on a remote monitor or display screen

In some embodiments, the present invention is a system for ultrasound imaging. The system includes a plurality of ultrasound transducer arrays, each housed in a single patch and comprising a plurality of ultrasound sensor elements and associated electronic; a plurality of beamformers, each electrically coupled to a respective ultrasound transducer array patch for combining outputs of said ultrasound sensor elements; a plurality of multiplexers, each electrically coupled to a respective beamformer for multiplexing the outputs of the plurality of beamformers; and a plurality of central processing unit (CPU), each electrically coupled to a respective multiplexer and configured to receive the combined outputs of said respective ultrasound transducer array, perform image processing and analytics and generate images or metrics to be displayed on a remote monitor or display screen.

The plurality of ultrasound transducer arrays may be configured to be individually placed on different locations on a subject and to be simultaneously used to generate images or metrics from said different locations.

The plurality of ultrasound sensor elements and/or the plurality of ultrasound transducer arrays are dynamically and selectively programmable to be active or inactive at a specific time or with a specific period.

### BRIEF DESCRIPTION OF THE DRAWINGS

FIG. 1 shows an exemplary sensor probe (patch), according to some embodiments of the present invention.

FIG. 2 shows an exemplary block of a probe (patch), according to some embodiments of the present invention.

FIG. 3 shows an exemplary detailed block diagram of the electronics of the system of the present invention.

FIG. 4 shows an exemplary block diagram of an exemplary beamforming circuit, according to some embodiments of the present invention.

FIG. 5 depicts a block diagram of an exemplary beamforming circuit, according to some embodiments of the present invention.

FIG. 6 is a simplified exemplary system block diagram, according to some embodiments of the present invention.

### DETAILED DESCRIPTION

In some embodiments, the present invention is directed to a system and method for performing automated, continuous and/or on-demand ultrasound imaging on medical patients, other subjects, or objects whereby medical personnel or other operators obtain multiple ultrasound images serially or simultaneously via a plurality of discrete sensors or probes placed on different locations on the subject's body or object's surface area. In some embodiments, the system of the present invention comprises combinations of miniature ultrasound array sensors strategically placed on a subject's body or on the surface of an object and thus enabling the ultrasound imaging to be performed at any time. The acquired ultrasound images, associated data, and metrics may then be sent to viewing stations electrically coupled (wired or wireless) to the system, to remote devices including mobile handheld devices, to electronic medical record (EMR) systems, and/or to picture archiving and communication systems (PACS). Metrics are data values periodically referenced by health care providers and represent one or more conditions of the patient, the monitoring equipment, and/or the environment. Some of the advantages of the present invention include being able to provide a non-invasive, image-based, continuous or on demand method of ultrasound diagnostics.

In some embodiments, the present invention includes a plurality of ultrasound transducer arrays, which can be phased, linear or curvilinear. In some embodiments, each transducer array is attached as a patch to the subject's skin or objects surface using a semi-permanent adhesive or mechanical fixture. Each transducer-array patch acquires ultrasound images and data of the subject's anatomy and physiological functions, such as those of the heart. In some embodiments, other types of probes for example, more invasive probes, such as transesophageal and intracardiac probes may be used. The probes may be of the 1-D, 1.25-D, 1.5-D, 1.75-D, 2-D, etc. array form factors where one of the typical uses of multidimensional arrays is for three-dimensional imaging or four-dimensional imaging where the fourth dimension is time.

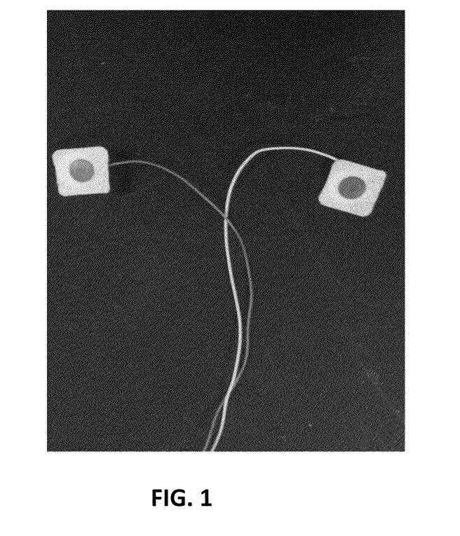

FIG. 1 shows two exemplary sensor/transducer array probes (patches), according to some embodiments of the present invention. The sensor array patch includes an array of ultrasonic elements housed in a single low profile patch and capable of producing and receiving ultrasound echoes, possibly a matching layer, and a backing layer. Each sensor array patch may include one or more transducer/sensor that transmit sound waves into the subject or object and pick up (receive) the reflections of the transmitted sound waves from the subject's body or object's surface. Each sensor array patch then transmits the received (reflection) signal to a central processing unit (CPU). Each sensor array patch may be equipped with an ultrasound beamformer and connected to a local CPU. In some embodiments, the patch probes have a roughly 1″×1″ footprint. They are attached using a strong medical grade adhesive similar to an ECG patch. The probes contain at least: an array of piezoelectric elements capable of sending and receiving ultrasonic signals, a backing for mechanical support and dampening of ultrasound waves, a housing to protect the array from damage, and a flexible circuit for electrical connection to the array.

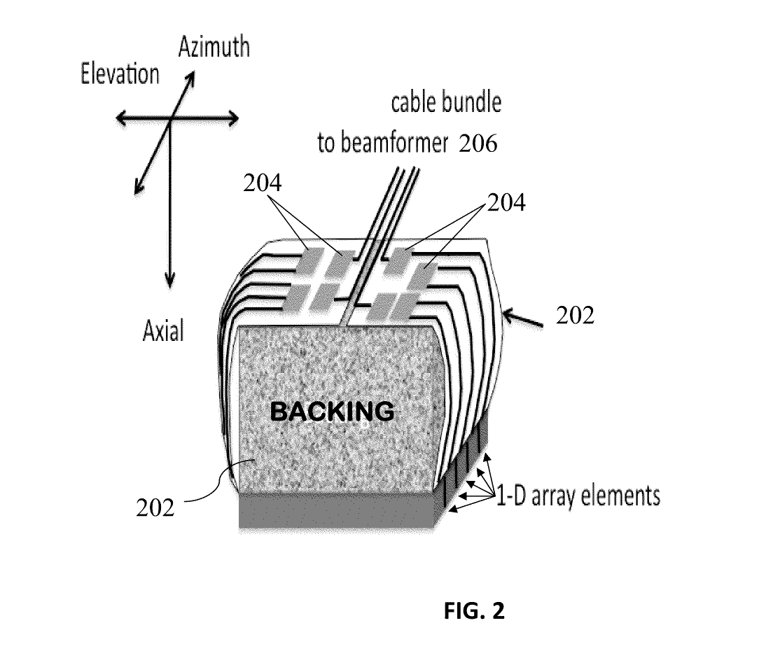

FIG. 2 shows a block of a probe (patch), according to some embodiments of the present invention. A thin flexible interconnect circuit 202 routes the connections from the arrays to the back of a backing 202. On the back of the backing 202, electronics 204 (components and/or ICs) are soldered or attached on this portion of the flexible interconnect circuit. If the array is a 1-D array, the patch may also have a fixed elevational lens to provide focusing in the out-of-plane dimension or direction. Because the array elements lie along one dimension only, this configuration is called a 1-D array. A 2-D array has elements in two dimensions—it would have a grid, a matrix, or a checkerboard pattern.

There could also be a flexible circuit to route electrical traces between elements and nearby electronics. A wire or cable connection bundle 206 with a wire connection to each sensor element outputs the output of each sensor element to components external to the array, for example, to a beamformer, as shown. Beamforming or spatial filtering may be used at both the transmitting and receiving ends of a phased array to combine elements in a phased array in such a way that signals at particular angles experience constructive interference while other signals experience destructive interference. Adaptive beamforming is used to detect and estimate the signal-of-interest at the output of a sensor array by least-squares spatial filtering and interference rejection. In some embodiments, the beamformer is inside of the single low profile patch along with the ultrasound sensor elements and associated electronics.

In some embodiments, the electronics includes pre-amplifiers, high-voltage protection, transmit/receive circuits, multiplexers, analog filters, demodulators, and multiplexer control logic using complex programmable logic devices (CPLDs) or field programmable gate arrays (FPGAs). In some embodiments, the array size may be roughly 1 cm in elevation and 1-2 cm in azimuth and the backing layer may be about 5-10 mm thick.

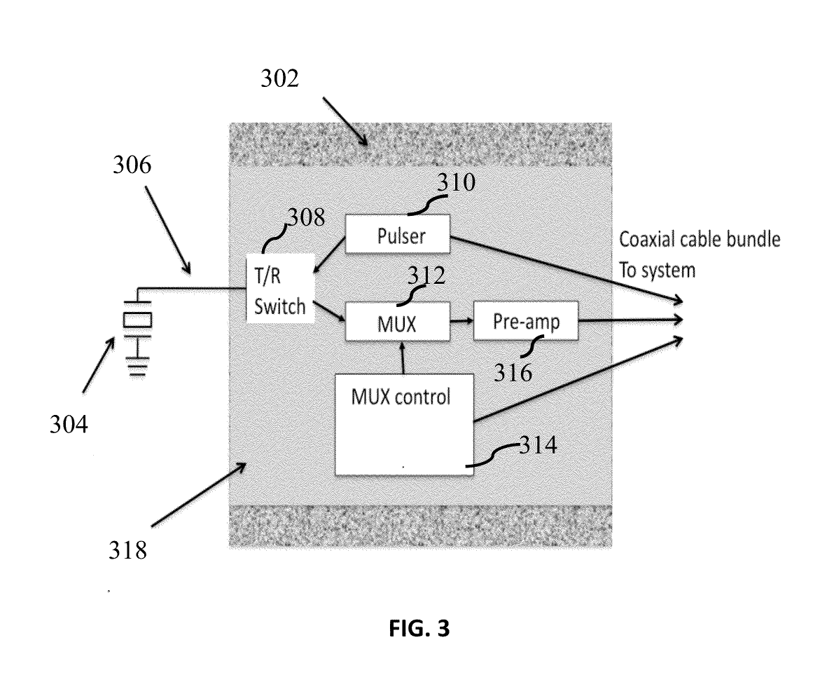

FIG. 3 shows an exemplary detailed block diagram of the electronics of the system of the present invention. In some embodiments, the electronics are located behind a backing layer 302. As shown, a transducer array element 304, located in the front of the backing, is connected to the electronics via a conducting trace 306 on the flexible circuit. In some embodiments, there is a transmit/receive (T/R) switch 308 to protect low voltage circuits from high voltage transients from a pulser 310. The pulser 310 typically transmits an 1-8 cycle electrical burst at a frequency within the pass-band of the ultrasound transducer. When this burst is applied to the transducer, it causes the transducer to mechanically vibrate thereby launching an ultrasound wave.

A multiplexer 312 and multiplexer controller 314 allow for selection of desired sensor elements and phasing or delaying for a given focus depth and direction. In some embodiments, the multiplexer controller 314 is programmable to allow for selection of specific sensors or set of sensors at a given time and other sensors at another time. In some embodiments, a preamplifier 316 conditions the signals to minimize signal loss across connectors, such as twisted pair and coaxial cables. In some embodiments, all or most of the sensor-related electronics are located on a flexible circuit 318. Alternatively, the electronics may be located on a printed circuit board (PCB) 318, which interfaces with a flexible circuit interconnect. Other different configurations of these electronics are possible depending on the type of beamformer used.

In some embodiments, each transducer-array patch is connected to an image processing unit 606. Within the image processing unit are a set of multiplexers located between the patches and the beamformer of the imaging system. In some embodiments, the multiplexers are configured in such a way to acquire several images from each transducer-array patch. The number of multiplexers normally equals the number of elements in each transducer array (probe or patch). In some embodiments, all the transducer arrays generally have the same number of transducer elements, although this is not necessary for the invention and other configurations may be used.

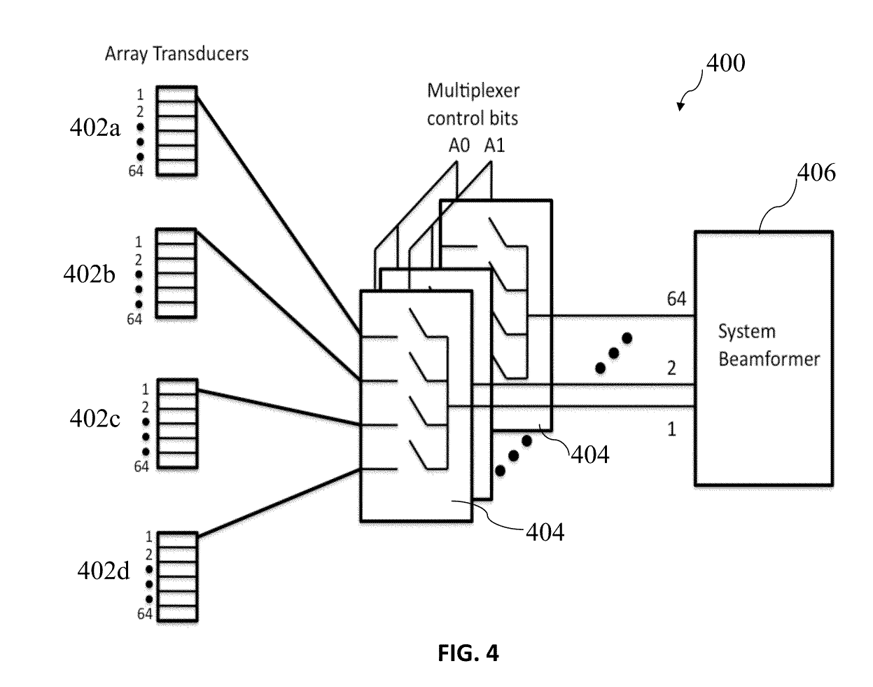

FIG. 4 shows a block diagram of an exemplary beamforming circuit 400, according to some embodiments of the present invention. As shown, there are four transducer-array patches 402a, 402b, 402c and 402d, each having 64 elements. Elements 1 from each transducer-array patch are the inputs into a 4:1 multiplexer 404 and there are 64 multiplexers 404, one for each element. In general, for these embodiments, an N:1 multiplexer is needed for N arrays. In some embodiments, the multiplexer control bits are the same for all 64 elements and therefore they can be electrically tied together. The output of the multiplexers 404 is the input to the ultrasound system front-end electronics and beamformer circuits 406. Accordingly, raw beamformed ultrasound data is generated at the output of 406. To make an image, data at the output of 406 is typically, band-pass filtered, demodulated (envelope detection), and log-compressed (or other forms of compression). To make an image, data at the output of 406 is typically, band-pass filtered demodulated (envelope detection), and log-compressed (or other forms of compression).

As an example, if four transducer-array patches each having 64 elements are used, only two control bits are needed to select one out of four multiplexers. In some embodiments, the two control bits are the same for all 64 multiplexers. In these embodiments, only a small number of digital multiplexer control signals are required since all the multiplexers share these same control signals. The beamforming can be done (by the beamformer) in stages with the advantages of a significant reduction in hardware and a corresponding reduction in manufacturing cost. The stages are comprised of clusters of elements, which are delayed (by respective delays) and summed by one or more adders. Afterwards, the sum of these clusters can be delayed and summed with other clusters of elements. For example, if 64 elements exist, 8 clusters, each cluster having 8 elements can first be digitized, delayed and summed. This would be the first stage. A second and final stage would be to delay and sum the signals from the 8 clusters. The time delay for each cluster can be determined by calculating the distance between the physical center of the cluster and the focal point. These delays can be applied in the digital domain by either shifting the data by an appropriate number samples based on the sampling frequency, use of an FIR filter, or both. If this first stage of beamforming is performed adjacent to the transducer-array patch, then fewer multiplexers are required to toggle between each transducer-array patch.

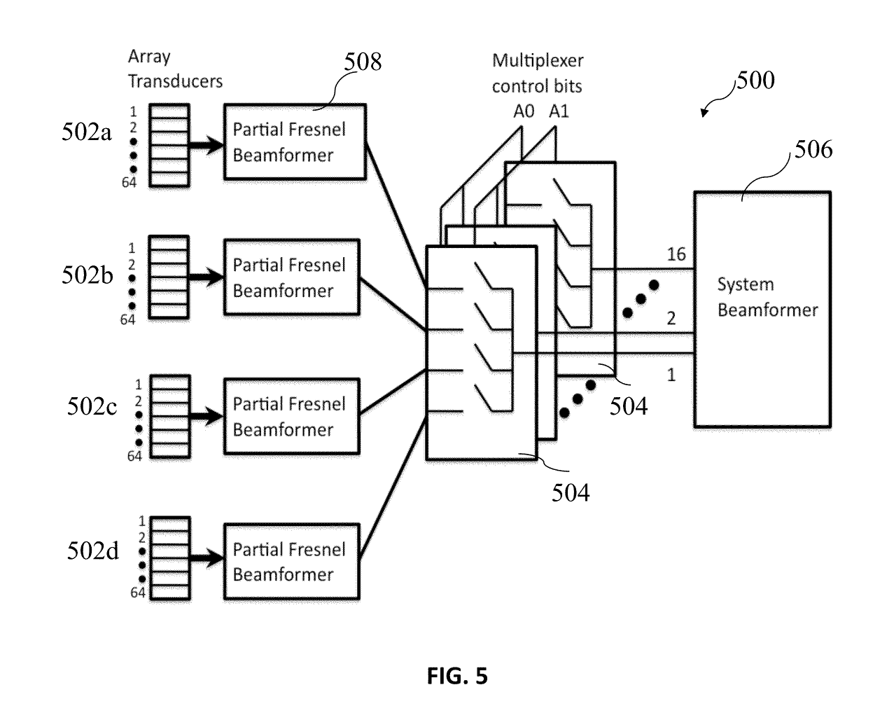

FIG. 5 depicts a block diagram of an exemplary beamforming circuit 500, according to some embodiments of the present invention. These embodiments, use Fresnel beamforming schemes to substantially reduce the number of required multiplexers. A detailed description of a Fresnel beamforming scheme is described in the U.S. Patent Pub. No. US2011/0060226 A1, the entire contents of which are herein expressly incorporated by reference. With this methodology, partial Fresnel beamforming is performed adjacent to each transducer-array patch to allow for a reduction in cabling complexity. For example, in FIG. 4, four sets of 64 cables/wires are required to output the outputs of the sensor elements, while in FIG. 5, four sets of 6-16 cables/wires are required when there is a 64-element array.

The partial Fresnel beamformers 508 lead to a reduction in the number of wires. If there are 64 array elements in each patch 502a-d, there are 64 signal, then only one from each element is beamformed. The partial Fresnel beamformers 508 combine the signals from the array elements and reduce the 64 signals into 6-16 signals, one connection for each signal. That is, going from partial Fresnel beamformers 508 to multiplexers 504, there are 6-16 connections. The system beamformer 506 performs the final beamforming, which delays and combines the 6-16 signals into a single signal at the output of the system beamformer 506.

In some embodiments, the partial Fresnel beamformer is built into each transducer-array patch. Because of this configuration, it is possible to establish the multiplexers such that there are multiple paths of signals so that ultrasound information is captured from multiple transducer-array patches simultaneously. This is particularly important for 3-D and 4-D image capture where volume acquisition rates can be slow.

Care is taken to not have the beams from simultaneously transmitting transducer-array patches overlap spatially, or, if they do, an appropriate signal processing method is used to resolve any acoustic/ultrasonic interference contributions from the simultaneously transmitting beams. That is, the acoustic/ultrasonic interferences are resolved to determine which echo signals came from which transducer. The signal processing methods may include simple time gating, or orthogonal coding of the transmitted waveforms and decoding of the received waveforms. During a calibration phase, the system emits a signal, for example a beep, should it not be able to resolve interference between simultaneously transmitting transducer-array patches.

In some embodiments, the target organ to be monitored is the heart of the subject. With the present invention, a diagnostician might desire to acquire images across several cardiac cycles. Several transducer-array patches are placed in suitable locations on the body of the subject. After several cardiac cycles have been acquired with one transducer-array patch, the multiplexers are reconfigured to acquire several more cardiac cycles from another transducer-array patch that provides a different view of the heart. This process is repeated until images from all transducer-array patches have been acquired and stored. This way, the doctor or technician can see and assess overall cardiac function and not just one part of heart such as, the left ventricle.

In some embodiments, images from all transducer-array patches are acquired at time intervals determined by a physician. In some embodiments, the time intervals and the particular transducer array(s) to be utilized may be programmed in the CPU. This way, there is no need for continuous presence of a technician/operator to turn the system on and off and to change the location of the probes.

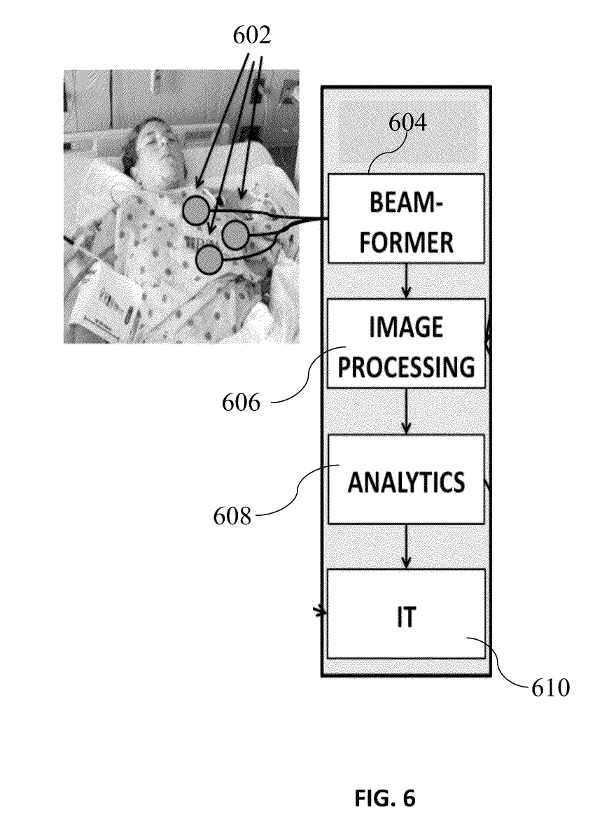

FIG. 6 is a simplified exemplary system block diagram, according to some embodiments of the present invention. As shown, the system includes patches/transducers 602, a beamformer module 604, an image processing module 606, an analytics module 608 and an information technology (IT) module 610. Some or all of above modules may be part of the CPU, or separate hardware modules. The beamformer module 604 performs all of the beamforming or the final stage of beamforming functions. This can include time gain control (TGC), time delay, phase shift, traditional apodization, envelope detection and/or demodulation of the signals. The beamformer module 604 can also be reconfigured to acquire Doppler flow information. The image processing module 606 improves image contrast and edge enhancement, among other image enhancing processes, to improve visualization. In some embodiments, the image processing module 606 performs one or more combination of frequency compounding, spatial compounding, log or gamma compression. The analytics module 608 extracts hemodynamic information from the processed images, using the know techniques. Examples of the functions of the analytics module are:

1. Identifying epicardial and endocardial borders in an automated or semi-automated fashion to estimate ventricular volumes in diastole and systole to calculate ejection fraction.

2. Tracking ventricular wall motion to identify ischemia.

3. Calculation of cardiac output based on measurement of outflow diameter and Doppler blood flow velocities.

The IT module 610 provides a layer of general data management commonly known in medical data management and IT fields. In addition, it securely stores the hemodynamic information and ultrasound images, and securely transmits this data to remote and/or local users, such as doctors, nurses and other members of the medical staff

In some embodiments, a CPU module provides the electronics, storage and network interconnectivity for some combination of modules 606, 608 and 608. The CPU may be configured to implement segmentation of subsets of transducer array patches where multiple images are acquired simultaneously. In these embodiments, the emitted ultrasound signals and echoes from different transducer array patches are each uniquely encoded and decoded (by an encoder and a decoder, or by the CPU) to avoid acoustic interference from different arrays, with a potentially faster cycle time, that is, to separate/isolate signals from different transducer arrays that are being used simultaneously.

In some embodiments, the beamformer 604 is a standard delay-and-sum beamformer or any other type of beamformer (e.g., analog, digital, hybrid). In some embodiments, the use of a Fresnel-based beamformer significantly reduces the thicknesses of the cable connecting each transducer-array patch, the associated hardware, and the number of multiplexers. The reduction is on the order of powers of 2; a four-way multiplex reduces hardware costs to 1 in 4, or 75%. Lowering the cost leads to the lowering of the financial barrier of acquiring ultrasound diagnostic equipment, leading to more lives being saved. Moreover, in some embodiments, the present invention utilizes new developments in miniaturization to reduce the amount of hardware required for the probe, cable and front-end electronics to enable new diagnostic techniques and protocols.

After the beamformer 604, the data propagates through a number of signal processing methods such as filtering, envelope detection, log compression, edge enhancement, and the kind, performed by the image processing module 606. Other signal/image processing methods and signal/image improvement methods such as dual apodization with cross-correlation (DAX), frequency compounding, spatial compounding, or any combination thereof may be used. DAX is a technique for sidelobe suppression where the transmit aperture or receive aperture is apodized by two different apodization functions. A more detailed description of DAX is provided in the U.S. Pat. No. 8,254,654, the entire contents of which are hereby expressly incorporated by reference.

In some embodiments, the data from the two apodization functions are cross-correlated, and the normalized cross-correlation coefficient reflects the amount of mainlobe versus clutter contribution in a signal. Signals with low cross-correlation coefficients are attenuated while signals with high cross-correlation coefficients are unchanged.

In some embodiments, the system of the present invention is also capable of performing color and pulsed-wave Doppler for obtaining flow information. To obtain flow information, multiple consecutive (for example, 2-16) transmit firings are repeated in the same spatial location. In general, one firing refers to a single point in time where the elements are excited to transmit ultrasound to a specific point in the field that you are imaging. For Doppler imaging, the same firing is repeated to get estimate blood flow. Transmit firings have more cycles (for example, 2-16 cycles) than what is used with grayscale B-mode imaging. The generated echoes from these repeated transmit firings are beamformed and then 1-D, 2-D cross-correlation, or phase shift estimation methods are performed on these echoes to estimate blood velocity at a specific location or region.

In some embodiments, the transmit firings for Doppler flow information are interspersed within the B-mode firings to enable duplex or triplex imaging. In duplex imaging with pulse-wave Doppler, the B-mode image and Doppler waveform are shown simultaneously but on different regions of the display. For duplex imaging with color Doppler, the estimate velocities are mapped to a false color map and overlayed on top of the B-mode image. Triplex imaging with B-mode, color and pulse-wave Doppler can also be done to perform multiple types of imaging nearly simultaneously, but at reduced frame rate.

After the signal and image processing stage, analytics are done to calculate hemodynamic and cardiac parameters such as ejection fraction, cardiac output, cardiac index, left ventricular end systolic volume, left ventricular end diastolic volume, and pulmonary artery systolic pressure, diameter of left ventricular outflow tract, velocity-time integral, tricuspid regurgitation, and peak systolic right ventricle-right atrium gradient. The multiple views from different area provide different hemodynamic information. For example, the parasternal long-axis view allows for measurement of the aortic diameter at the left ventricle. Combining this diameter with a Doppler velocity obtained from the apical five-chamber view allows calculation of cardiac output, one form of hemodynamic function. In another example, a parasternal short-axis view is used for assessing left ventrical function, namely ejection fraction—another hemodynamic parameter.

In some embodiments, software routines, sometime with the help of medical personnel, identify wall motion abnormalities, regurgitant flow, valvular abnormalities or rupture, fluid responsiveness, fluid status, stenosis, tamponade, ischemia, myocardial infarction pericardial fluid, and right ventricular function, from the multiple (ultrasonic) images/data. These images and analytics are then printed on hardcopy or transferred to a nurse station, a data network, and/or a cloud for viewing by physicians, cardiologists, radiologists, or other users.

In some embodiments, the new probe configuration of the present invention, which uses one or more low-profile transducer-array patches allows medical personnel to acquire all necessary views of an organ or multiple organs automatically, and/or periodically or on demand, and to do so without having to send someone to monitor the subject and relocate the probe to manually run a probe on the subject's body.

Furthermore, the low-profile transducer array patches of the present invention are immediately reconfigurable “on-the-fly” to suit the situation of diagnostics or treatment. For example, the CPU can be programmed to utilize probes 1, 3, and 5, every 5 minutes and probes 2 and 4 every 30 minutes to collect the respective images. Also, additional transducer array patches may be dynamically added or removed as needed, without requiring the system to be reset or restarted. In some embodiments, the system (e.g., the IT module 610 or the CPU) maintains a respective proper history of previously acquired images and data across all changes in configuration.

If the probes/patches are 2-D arrays, interrogation of a 3-D volume can be done rapidly allowing for 4-D imaging, the fourth dimension being time. Each 2-D array probe constructs its own 3-D or 4-D image. That is, 1-D arrays allow for steering and focusing within a single plane and 2-D arrays allow for steering and focusing of the beam within a volume.

In some embodiments, the probes are strategically placed on the desired areas (of subject's body or object's surface) and the best 2-5 images/views are selected for a more simplified analysis. In some embodiments, at least two substantially different images/views, such as parasternal view, an apical view and/or a subcostal view, are selected for analysis.

In some embodiments, the present invention is a system for performing automated, continuous or on-demand ultrasound imaging on subjects or objects. The system includes one or more low-profile ultrasound transducer-array patches. Each transducer-array patch may include one or more transducer that transmit sound waves into the subject or object and picks up (receives) the reflections of the transmitted sound waves from the subject's body or object's surface. Each transducer-array patch is equipped with an ultrasound beamformer and is connected to a local central processing unit (CPU). The CPU is electrically coupled to remote view stations directly or over a wired or wireless network. Similarly, the ultrasound transducer-array patches are dynamically reconfigurable and selectable and may be coupled to the CPU with or without wires.

In some embodiments, the present invention is a method for performing automated, continuous or on-demand ultrasound imaging on subjects or objects and therefore providing operators with improved diagnostics on a subject or an object. The method uses a plurality of low-profile ultrasound transducer-array patches that are individually placed in different locations on the subject or subject. The transducer-array patches are enabled to transmit sound waves onto the subject or subject and receive the reflections of the sound waves. The reflected sound wave data is then transmitted to a central processing unit (CPU), and the CPU then sends the sound wave information to remote viewing stations, for example, EMR and/or PACs. The sound waves are in the ultrasound frequency range and are digitized for further processing. Optionally, the sound waves may be compressed after being processed and before being transmitted to the remote viewing stations.

It will be recognized by those skilled in the art that various modifications may be made to the illustrated and other embodiments of the invention described above, without departing from the broad inventive scope thereof. It will be understood therefore that the invention is not limited to the particular embodiments or arrangements disclosed, but is rather intended to cover any changes, adaptations or modifications which are within the scope and spirit of the invention as defined by the appended claims.

What is claimed is:

1. A system for ultrasound imaging comprising:
an ultrasound transducer array housed in a single patch and comprising a plurality of ultrasound sensor elements and associated electronics;
a beamformer electrically coupled to the ultrasound transducer array patch for combining outputs of said ultrasound sensor elements; and
a central processing unit (CPU) electrically coupled to the beamformer and configured to receive the combined outputs of said ultrasound sensor elements, perform image processing and analytics and generate an image or metrics to be displayed on a remote display screen.

2. The system of claim 1, wherein the beamformer is wirelessly coupled to the CPU.

3. The system of claim 1, wherein each of said plurality of ultrasound sensor elements are dynamically and selectively programmable to be active or inactive at a specific time or with a specific period.

4. The system of claim 1, further comprising a plurality of first and second ultrasound transducer arrays housed in a single first and a single second patches, respectively , a plurality of first and second beamformers, and a plurality of first and second CPUs, wherein said plurality of ultrasound transducer arrays, and said plurality of first and second ultrasound transducer arrays are configured to be individually placed on different locations on a subject.

5. The system of claim 4, further comprising encoding means to separate or isolate signals from said plurality of first and second ultrasound transducer arrays that are being used simultaneously.

6. The system of claim 1, wherein said associated electronics of said ultrasound transducer array comprise:
a pulser to transmit ultrasound energy;
a transmit/receive (T/R) switch to protect low voltage circuits from high voltage transients from the pulser;
a multiplexer for multiplexing the outputs of said ultrasound sensor elements;
a multiplexer controller for controlling the multiplexer to select one of its inputs and an output and configured to be programmable to select a desired ultrasound sensor element from the plurality of ultrasound sensor elements; and
a preamplifier to condition the outputs of said ultrasound sensor elements.

7. The system of claim 1, wherein said beamformer is configured to perform one or more of time gain control, time delay, phase shift, apodization, envelope detection, and signal demodulation.

8. The system of claim 1, wherein said beamformer includes a plurality of delay circuits to delay respective signals from clusters of ultrasound sensor elements and an adder to add the delayed signals to perform beamforming in stages.

9. The system of claim 1, wherein said beamformer is a Fresnel beamformer.

10. The system of claim 1, wherein said single patch further comprises:
a backing;
a thin flexible interconnect circuit to route connections from the plurality of ultrasound sensor elements; to the backing; and
a connection bundle electrically coupled to each of the plurality of ultrasound sensor elements to output outputs of each sensor element.

11. The system of claim 1, wherein said ultrasound transducer array is a 2-dimensional array housed in said single patch and configured to interrogate a 3-dimensional volume, and wherein said CPU is configured to generate 3-dimensional images over a programmable period of time.

12. A method for ultrasound imaging comprising:
placing a plurality of ultrasound imaging system according to claim 1 on different locations on a subject;
capturing a plurality of ultrasound images from said different locations; and
transmitting said captured images to a remote location.

13. A system for ultrasound imaging comprising:
a plurality of ultrasound transducer arrays, each housed in a single patch and comprising a plurality of ultrasound sensor elements and associated electronic;
a plurality of beamformers, each electrically coupled to a respective ultrasound transducer array patch for combining outputs of said ultrasound sensor elements;
a plurality of multiplexers, each electrically coupled to a respective beamformer for multiplexing the outputs of the plurality of beamformers; and
a plurality of central processing unit (CPU), each electrically coupled to a respective multiplexer and configured to receive the combined outputs of said respective ultrasound transducer array, perform image processing and analytics and generate an image or metrics to be displayed on a remote display screen.

14. The system of claim 13, wherein said plurality of ultrasound transducer arrays are configured to be individually placed on different locations on a subject and to be simultaneously used to generate images or metrics from said different locations.

15. The system of claim 14, further comprising encoding means to separate or isolate signals from said plurality of ultrasound transducer arrays that are being used simultaneously.

16. The system of claim 13, wherein each of said plurality of ultrasound sensor elements are dynamically and selectively programmable to be active or inactive at a specific time or with a specific period.

17. The system of claim 13, wherein each of said plurality of ultrasound transducer arrays are dynamically and selectively programmable to be active or inactive at a specific time or with a specific period.
